(19) United States
(12) Patent Application Publication
(10) Pub. No.: US 2007/0104806 A1
(43) Pub. Date: May 10, 2007

(54) Compositions containing cotinus coggygria extract and use thereof in treating hemorrhoids

(76) Inventors: Miri Seiberg, Princeton, NJ (US); Violetta Iotsova Stone, Robbinsville, NJ (US); Renbin Zhao, Plainsboro, NJ (US); Elizabeth Bruning, Somerset, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/590,563

(22) Filed: Oct. 31, 2006

Related U.S. Application Data
(63) Continuation-in-part of application No. 11/387,892, filed on Mar. 23, 2006.
Application No. 11/387,892 is a continuation-in-part of application No. 11/313,079, filed on Dec. 20, 2005.
Application No. 11/313,079 is a continuation-in-part of application No. 11/248,465, filed on Oct. 12, 2005.
Application No. 11/248,465 is a continuation-in-part of application No. 10/973,313, filed on Oct. 26, 2004.

(51) Int. Cl. A61K 36/185; A61K 36/48; A61K 36/28

(52) U.S. Cl. 424/725; 424/764; 424/757; 424/769

(57) ABSTRACT

The present invention relates to a method for treating hemorrhoids by topically administering to tissue in need of such treatment a composition containing cotinus coggygria extract.

### CROSS REFERENCE TO RELATED APPLICATIONS

This is a continuation-in-part of co-pending application Ser. No. 11/387,892, filed Mar. 23, 2006, which is a continuation-in part of co-pending application Ser. No. 11/313,079, filed Dec. 20, 2005, which is a continuation-in-part of co-pending application Ser. No. 11/248,465, filed Oct. 12, 2005, which was a continuation-in-part of co-pending application Ser. No. 10/973,313, filed Oct. 26, 2004, which are hereby incorporated in their entirety.

### BACKGROUND OF THE INVENTION

Hemorrhoids are a condition in which the veins around the anus or lower rectum are swollen and inflamed. Hemorrhoids may result from straining to move stool, pregnancy, aging, and chronic constipation. Other etiologies include sedentary life, tight-laced clothes, climate and seasons, and the intrinsic weakness of the blood vessel wall. The most common symptom of internal hemorrhoids is bright red blood covering the stool, on toilet paper, or in the toilet bowl. However, an internal hemorrhoid may protrude through the anus outside the body, becoming irritated and painful. A large percent of the population has hemorrhoids by age 50, and they are common among pregnant women.

Hemorrhoids have anatomical alterations, causing blood vessels that lie beneath the anal mucosa and perianal skin to dilate. In addition, the muscular and connective tissue supports of these blood vessels become damaged and broken, resulting in the downward displacement of hemorrhoidal cushions. The collagen and elastin support of the anorectal region weaken and break, causing hemorrhoidal cushions to lose their support and become displaced. See, Han et al., Zhonghua Wei Chang Wai Ke Za Zhi (2005) 8(1):56-9 and Serge D., Ann Ital Chir (1995)66(6):747-50.

Biochemical studies had identified enhanced levels of elastin-degrading enzymes such as MMP-2 and MMP-9 in hemorrhoidal tissues, which could account for the degraded support network. Lierse W, Langenbecks Arch Chir Suppl II Verh Dtsch Ges Chir (1989) 769-72.

Treatments of hemorrhoids are used initially to relieve symptoms, and may include warm baths, and topical hemorrhoidal creams or suppositories, including retinol, which increases collagen at the affected area. However, in severe cases, hemorrhoids may need to be surgically removed. Thus, there still remains a need for new and improved treatments, modes of prevention, and for reducing the risk of or the severity of hemorrhoids.

Cotinus coggygria extract is traditionally believed to be useful as an anti-microbial treatment, used in the form of external washes. See, e.g., US Patent Applications Nos. 2002/0132021 where the extract is mentioned to be active against E.coli, Staphylococcus aureus and S. cerevisiae, as well as having anti-cancer activity. The dried leaf and twig of Cotinus coggygria is used in Chinese traditional medicine to eliminate “dampness” and “heat”, and as an antipyretic (Huang K. C., The Pharmacology of Chinese Herbs (CRS Press, 1999, pp 193-194). A yellow/orange dye can be obtained from the root and stem and can be used for fabric dying. The leaves and bark are a good source of tannins (Grieve M. A Modern Herbal. Dover Publications, Inc. NY, 1971, pp 779-781).

The present invention relates to the unexpected discovery that Cotinus coggygria extract and its combinations with other extracts or agents are effective for preventing, reducing the risk and reducing the severity of symptoms of hemorrhoids.

### SUMMARY OF THE INVENTION

In one aspect, the present invention relates to a method for treating hemorrhoids by topically administering to tissue in need of such treatment a composition containing cotinus coggygria extract.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

### DEFINITIONS

What is meant by “treating a hemorrhoid” is inhibiting or slowing the formation of hemorrhoids, reducing the severity or pain of hemorrhoids, and/or enhancing the healing of the hemorrhoids.

What is meant by a “product” is a product in finished packaged form. In one embodiment, the package is a container such as a plastic, metal or glass tube or jar containing the composition. The product may further contain additional packaging such as a plastic or cardboard box for storing such container. In one embodiment, the product contains instructions directing the user to administer the composition to the area in need of such treatment (e.g., the area around the anus) to treat hemorrhoids. Such instructions may be printed on the container, label insert, or on any additional packaging.

What is meant by “promoting” is promoting, advertising, or marketing. Examples of promoting include, but are not limited to, written, visual, or verbal statements made on the product or in stores, magazines, newspaper, radio, television, internet, and the like. Examples of such statements include, but are not limited to, “decreases the risk of hemorrhoids”, “prevents or reduces pain and discomfort from hemorrhoids”, “reduces the severity of hemorrhoids”, “treats hemorrhoids,” and “enhances hemorrhoid healing.

As used herein, “administering” means contacting the tissue, e.g., by use of the hands or an applicator such, but not limited to, a water-insoluble substrate such as a wipe, tube, roller, spray, patch, bandage, dropper, and suppository.

As used herein, “composition” means a composition suitable for administration to the tissue (e.g., skin or mucosal tissue).

As used herein, “cosmetically-acceptable” means that the ingredients which the term describes are suitable for use in contact with tissues (e.g., the skin or mucosal tissue) without undue toxicity, incompatibility, instability, irritation, allergic response, and the like.

As used herein, “safe and effective amount” means an amount of the extract or of the composition sufficient to induce the treatment of hemorrhoids, but low enough to avoid serious side effects. The safe and effective amount of the compounds or composition will vary with the area being treated, the age, health and skin type of the end user, the duration and nature of the treatment, the specific extract, ingredient, or composition employed, the particular cosmetically-acceptable carrier utilized, and like factors.

Cotinus Coggygria Extract

What is meant by a “Cotinus coggygria extract” is a blend of compounds isolated from a Cotinus coggygria plant. In one embodiment, the compounds are isolated from the leaf of the plant. In a further embodiment, the compounds are isolated from dried leaves of the plant. Such compounds may be isolated from one or more parts of the plant (e.g., the whole plant, flower, seed, root, rhizome, bark, wood, stem, fruit and/or leaf of the plant) by physically removing a piece of such plant, such as grinding a root of the plant. Such compounds may also be isolated from the plant by using extraction procedures well known in the art (e.g., the use of organic solvents such as lower C₁-C₈ alcohols, C₁-C₈ alkyl polyols, C₁-C₈ alkyl ketones, C₁-C₈ alkyl ethers, acetic acid C₁-C₈ alkyl esters, and chloroform, and/or inorganic solvents such as water, inorganic acids such as hydrochloric acid, and inorganic bases such as sodium hydroxide). In one embodiment, the Cotinus coggygria extract contains only hydrophilic compounds (e.g., isolated by using a hydrophilic solvent, such as water or ethanol). In one embodiment, the Cotinus coggygria extract is an aqueous extract from the leaf of Cotinus coggygria.

In one embodiment, the composition contains a safe and effective amount of the Cotinus coggygria extract. In one embodiment, the extract is present in the composition in an amount from about 0.001% to about 20% by weight, in particular in an amount from about 0.01% to about 10% by weight. Unless stated otherwise, the weight of the extract refers to the dry weight of the extract.

Malva Sylvestris Extract

What is meant by a “Malva sylvestris extract” is a blend of compounds isolated from the plant Malva sylvestris. In one embodiment, the compounds are isolated from the flowers of the plant. In a further embodiment, the compounds are isolated from dried flowers of the plant. Such compounds may be isolated from one or more part of the plant (e.g., the whole plant, flower, seed, root, rhizome, stem, fruit and/or leaf of the plant) by physically removing a piece of such plant, such as grinding a flower of the plant. Such compounds may also be isolated from the plant by using extraction procedures well known in the art (e.g., the use of organic solvents such as lower C₁-C₈ alcohols, C₁-C₈ alkyl polyols, C₁-C₈ alkyl ketones, C₁-C₈ alkyl ethers, acetic acid C₁-C₈ alkyl esters, and chloroform, and/or inorganic solvents such as water, inorganic acids such as hydrochloric acid, and inorganic bases such as sodium hydroxide). In one embodiment, the Malva sylvestris extract contains only hydrophilic compounds (e.g., isolated by using a hydrophilic solvent, such as water or ethanol). In one embodiment, the Malva sylvestris is extract is an aqueous extract from the flowers.

In one embodiment, the composition contains a safe and effective amount of the Malva sylvestris extract. In one embodiment, the extract is present in the composition in an amount from about 0.001% to about 20% by weight, in particular in an amount from about 0.01% to about 10% by weight. Unless stated otherwise, the weight of the extract refers to the dry weight of the extract.

Legume Extract

What is meant by a “legume extract” is a blend of compounds isolated from a legume fruit. A legume is a plant from the family Leguminosae, which has a dehiscent fruit such as a bean, pea, or lentil. Examples of legume fruits, include but are not limited to, beans such as soybeans, lentil beans, peas, and peanuts. The legume extract may contain the entire legume fruit (e.g., the legume fruit ground into a powder) or only a portion of the legume. The legume extract may be in the form of a fluid (e.g., a mixture of the legume fruit and water) or a solid (e.g., legume fruits powders).

In one embodiment, the composition contains a safe and effective amount of the legume extract. In one embodiment, the extract is present in the composition in an amount from about 0.001% to about 20% by weight, in particular in an amount from about 0.01% to about 10% by weight. Unless stated otherwise, the weight of the extract refers to the dry weight of the extract.

In one embodiment, the legume extract is a soybean extract. The soybean extract may contain only a portion of the soybean (e.g., an extract of the soybean such as a lipid reduced soybean powder or filtered soymilk) or may contain the entire soybean (e.g., a ground powder of the legume). The soy extract may be in the form of a fluid (e.g., soymilk) or a solid (e.g., a soybean powder or soymilk powder).

In one embodiment the soybean extract contains all the ingredients naturally found in soybeans, at the relative concentrations as found in the beans, with exception of water content. In another embodiment, the soybean extract is a non-denatured soybean extract. “Denaturation” is defined in the Bantam Medical Dictionary (1990 edition) as “the change in the physical and the physiological properties of a protein. Such changes are brought about by heat, X-rays or chemicals such as ethanol and other organic solvents, or detergents. These changes include loss of activity (in the case of enzymes or enzyme inhibitors) and loss (or alteration) of antigenicity (in the case of antigens)”.

What is meant by “non-denatured soybean extract” is a soybean extract in which the processing for the derivation of such soybean extract (e.g., the temperature, extraction media) did not eliminate its protease inhibitory activity. In one embodiment, the non-denatured state of the soybean extract of this invention is measured by the presence of an intact soybean trypsin inhibitor (STI) protein. In another embodiment it is measured by the presence of trypsin inhibitory activity.

In one embodiment, the soybean extract is soybean powder. Soybean powder may be made by grinding dry soybeans. In one embodiment, the soybean powder has a moisture content of less than about 10% such as less than about 5%. In one embodiment, the soybean powder is lyophilized. In one embodiment, the soybean extract is soymilk or soymilk powder. Soymilk is a combination of solids derived from soybeans and water, the mixture of which has some or all of the insoluble constituents filtered off. Soymilk powder is evaporated soymilk, which in one embodiment, is in a lyophilized or spray-dried form.

Other Extracts

In one embodiment, the compositions of the present invention contain one or more of the extracts from plants selected from the group consisting of Matricaria chamomilla, Matricaria recutita, Thymus vulgaris, Thymus serpyllum, Arctostaphylos uva-ursi, and plant-extracts containing isoflavonoids or oligo procyannidins such as pine bark, grape, bilberry, cranberry, black currant, green tea, black tea, and yukinoshita extract. In one embodiment, the composition contains a safe and effective amount of one or more of such extracts. In one embodiment, the extract is present in the composition in an amount from about 0.001% to about 20% by weight, in particular in an amount from about 0.01% to about 10% by weight. Unless stated otherwise, the weight of the extract refers to the dry weight of the extract.

Compositions

The compositions useful in the present invention involve formulations suitable for administering to the target tissues. In one embodiment, the composition contains a safe and effective amount of (i) Cotinus coggygria extract and (ii) a cosmetically-acceptable carrier. In one embodiment, the cosmetically-acceptable carrier is from about 50% to about 99.99%, by weight, of the composition (e.g., from about 80% to about 99%, by weight, of the composition).

The compositions may be made into a wide variety of product types that include but are not limited to solutions, suspensions, lotions, creams, gels, sticks, sprays, ointments, cleansing liquid washes and solid bars, pastes, foams, powders, wipes, patches, hydrogels, film-forming products, liquid drops, suppositories, and the like. These product types may contain several types of cosmetically-acceptable carriers including, but not limited to solutions, suspensions, emulsions such as microemulsions and nanoemulsions, gels, solids and liposomes. The following are non-limitative examples of such carriers. Other carriers can be formulated by those of ordinary skill in the art.

The compositions useful in the present invention can be formulated as solutions. Solutions typically include an aqueous or organic solvent (e.g., from about 50% to about 99.99% or from about 90% to about 99% of a cosmetically-acceptable aqueous or organic solvent). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, and mixtures thereof.

A lotion can be made from such a solution. Lotions typically contain from about 1% to about 20% (e.g., from about 5% to about 10%) of an emollient(s) and from about 50% to about 90% (e.g., from about 60% to about 80%) of water. As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7^{th} Edition, 1997) (hereinafter “ICI Handbook”).

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s). Examples of thickening agents include, but are not limited to, those set forth in the ICI Handbook pp. 1693-1697.

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, wipe containing powder, and suppository). Excipients to be used in formulating suppositories include, but are not limited to, semi-synthetic glycerides and polyethylene glycols and optionally emulsifiers and surfactants.

The compositions useful in the subject invention may contain, in addition to the aforementioned components, a wide variety of additional oil-soluble materials and/or water-soluble-materials conventionally used in compositions for use on skin and mucosal tissues at their art-established levels.

Additional Hemorrhoids-Affecting Agents

In one embodiment, the present invention further includes one or more hemorrhoid-affecting agents. Examples of hemorrhoid-affecting agents include, but are not limited to, (i) vasoconstrictors such as ephedrine, epinephrine, and phenylephrin, (ii) skin protectants such as witch hazel, mineral oil, petrolatum, shark liver oil, glycerin, kaolin, lanolin, wood alcohol, zinc oxide, and shark liver oil, (iii) local anesthetics and analgesics such as pramoxine, (iv) anti-itch actives such as hydrocortisone,(v) live yeast cell extracts such as Saccharomyces Ferment Lysate Filtrate, and (vi) anti-inflammatory agents such as tocopherols and extracts such as Feverfew extract.

Additional Cosmetically Active Agents

In one embodiment, the composition further contains another cosmetically active agent in addition to the extracts. What is meant by a “cosmetically active agent” is a compound (e.g., a synthetic compound or a compound isolated from a natural source, or a natural extract containing a mixture of compounds) that has a cosmetic or therapeutic effect on the tissue, including, but not limiting to, flavonoids, isoflavonoids, oligomeric procyannidins, anti-microbial agents such as anti-yeast agents, anti-fungal, and anti-bacterial agents, anti-inflammatory agents, anti-parasite agents, external analgesics, antioxidants, keratolytic agents, detergents/surfactants, moisturizers, nutrients, vitamins, minerals, energy enhancers, firming agents, agents for skin conditioning, and odor-control agents such as odor masking or pH-changing agents. The cosmetically active agent will typically be present in the composition of the invention in an amount of from about 0.001% to about 20% by weight of the composition, e.g., about 0.005% to about 10% such as about 0.01% to about 5%.

Examples of vitamins include, but are not limited to, vitamin A, vitamin Bs such as vitamin B3, vitamin B5, and vitamin B12, vitamin C, vitamin K, vitamin E such as alpha, gamma or delta-tocopherol, and derivatives and mixtures thereof.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), different types of tocopherols (e.g., alpha-, gamma-, and delta-tocopherols and their esters such as acetate) and their mixtures, tocotrienols, and ubiquinone. Natural extracts containing antioxidants suitable for use in the compositions of this invention, include, but not limited to, extracts containing flavonoids, isoflavonoids, and their derivatives such as genistein and daidzein (e.g., such as Soy and Clover extracts, extracts containing resveratrol and the like) or oligomeric procyannidins. Examples of such natural extracts include grape seed, green tea, pine bark, and propolis.

Other Materials

Various other materials may also be present in the compositions useful in the subject invention. These include humectants, proteins and polypeptides, preservatives and an alkaline agent. Examples of such agents are disclosed in the ICI Handbook, pp. 1650-1667. The compositions of the present invention may also contain chelating agents (e.g., EDTA) and preservatives (e.g., parabens). Examples of suitable preservatives and chelating agents are listed in pp. 1626 and 1654-55 of the ICI Handbook. In addition, the compositions useful herein can contain conventional cosmetic adjuvants, such as colorants such as dyes and pigments, opacifiers (e.g., titanium dioxide), and fragrances.

Mineral Water

The compositions of the present invention may be prepared using a mineral water, for example mineral water that has been naturally mineralized such as Evian® Mineral Water (Evian, France). In one embodiment, the mineral water has a mineralization of at least about 200 mg/L (e.g., from about 300 mg/L to about 1000 mg/L). In one embodiment, the mineral water contains at least about 10 mg/L of calcium and/or at least about 5 mg/L of magnesium.

Water-Insoluble Substrate

In one embodiment, the composition is administered by a water-insoluble substrate. By “water-insoluble” is meant that the substrate, upon immersion in distilled water at 25° C., does not readily dissolve in or readily break apart. Under such immersion, while portions of the water-insoluble substrate may be leachable or readily soluble in the distilled water, at least another portion of the water-insoluble substrate remains intact. For example, the other portion may be readily manipulated, such as picked up and transported as an interconnected cohesive unit, by a user's hands. In an alternative embodiment of the invention, the water-insoluble substrate may, however, be disintegrated and/or dissolved slowly in the distilled water, i.e., over a period of several hours up to several days.

A wide variety of materials can be used as the water-insoluble substrate. Examples of suitable substrates include, but are not limited to, fibrous substrates such as substrates including or formed from non-woven fibers, woven fibers, hydro-entangled fibers, or air-entangled fibers. The water-insoluble substrate may include natural sponges; synthetic sponges, and polymeric netted meshes.

The water-insoluble substrate may be formed to retain a liquid impregnate containing the extract(s) (such as by absorbing the liquid impregnate among, along, and/or between fibers comprising the water-insoluble substrate) for a period of time at least as long as from when the product is manufactured to a time when the product is used by a consumer (i.e., a shelf storage period). In this embodiment of the invention, during this shelf storage period the water-insoluble substrate should generally maintain its mechanical integrity such that a user can apply the water-insoluble substrate to the skin and transferring liquid impregnate thereto.

The water-insoluble substrate may be flushable. As used herein, by “flushable” is meant that the substrate will pass through at least 10 feet of waste pipe in two toilet flushes. The material may also be biodegradable.

In one embodiment of the invention, the substrate includes a non-woven material. By “non-woven” is meant that the substrate, or a layer of the substrate, is comprised of fibers that are not woven into a fabric but rather are formed into a sheet, mat, or pad layer. The fibers can either be random (i.e., randomly aligned) or they can be carded (i.e., combed to be oriented in primarily one direction. Furthermore, the non-woven substrate can be composed of a combination of layers of random and carded fibers).

Non-woven substrates may be comprised of a variety of natural and/or synthetic materials. By “natural” it is meant that the materials are derived from plants, animals, insects, or byproducts of plants, animals, and insects. By “synthetic” it is meant that the materials are obtained primarily from various man-made materials or from natural materials, which have been further altered. Non-limiting examples of natural materials useful in the present invention are silk fibers, keratin fibers (such as wool fibers, camel hair fibers) and cellulosic fibers (such as wood pulp fibers, cotton fibers, hemp fibers, jute fibers, and flax fibers).

Examples of synthetic materials include, but are not limited to, those selected from the group containing acetate fibers, acrylic fibers, cellulose ester fibers, cotton fibers, modacrylic fibers, polyamide fibers, polyester fibers, polyolefin fibers, polyvinyl alcohol fibers, rayon fibers, polyurethane foam, and mixtures thereof.

Substrates made from one ore more of the natural and synthetic materials useful in the present invention can be obtained from a wide variety of commercial sources such as Freudenberg & Co. (Durham, N.C. USA), BBA Nonwovens (Nashville, Tenn. USA), PGI Nonwovens (North Charleston, S.C. USA), Buckeye Technologies/Walkisoft (Memphis, Tenn. USA), Sansho Shigyo K.K. (Tosa City, Kouchi, Japan), and Fort James Corporation (Deerfield, Ill. USA).

Methods of making non-woven substrates are also well known in the art. Such methods include, but are not limited to, air-laying, water-laying, melt-blowing, spin-bonding, or carding processes. The resulting substrate, regardless of its method of production or composition, is then generally subjected to at least one of several types of bonding operations to anchor the individual fibers together to form a self-sustaining web. The non-woven substrate can be prepared by a variety of processes including hydro-entanglement, thermally bonding, chemical bonding and combinations of these processes. Moreover, the substrates can have a single layer or multiple layers. In addition, a multi-layered substrate can include film layer(s) (e.g., aperture or non-aperture film layers) and other non-fibrous materials.

Strength or firmness of the non-woven material may be a desirable attribute. This can be achieved, for example, by the addition of binding materials, such as wet strength resins, or the material may be made of polymer binder coatings, stable fibres, e.g. based on cotton, wool, linen and the like. Examples of wet strength resins include, but are not limited to, vinyl acetate-ethylene (VAE) and ethylene-vinyl chloride (EVCL) Airflex emulsions (Air Products, Lehigh, Pa.), Flexbond acrylic polymers (Air Products, Lehigh, Pa.), Rhoplex ST-954 acrylic binder (Rohm and Haas, Philadelphia, Pa.), and Ethylene-vinyl acetate (EVA) emulsion (DUR-O-SET® by National Starch Chemicals, Bridgewater, N.J.). The amount of binding material in the substrate may range from about 5% to about 20%, by weight, of the substrate.

Non-woven materials of increased strength can also be obtained by using the so-called spunlace or hydro-entanglement technique. In this technique, the individual fibers are twisted together so that an acceptable strength or firmness is obtained without the need to use binding materials. The advantage of the latter technique is the excellent softness of the non-woven material.

The basis weight of the water-insoluble substrate may range from about 10 grams per square meter (gsm) to about 100 gsm, such as between about 30 gsm and about 70 gsm. The water-insoluble substrate may have an average thickness that is less than about 5 mm, such as between about 0.1 mm and about 1 mm.

In one embodiment of the invention, the non-woven material includes or is made from a superabsorbent polymer. For the purposes of the present invention, the term “superabsorbent polymer” refers to materials which are capable of absorbing and retaining at least about 10 times their weight in water under a 0.5 psi pressure. The superabsorbent polymer particles of the invention may be inorganic or organic crosslinked hydrophilic polymers, such as polyvinyl alcohols, polyethylene oxides, crosslinked starches, guar gum, xanthan gum, and other material known to the art of absorbent article manufacture.

Additives may also be added in order to increase the softness of the substrates. Examples of such additives include, but are not limited to, polyols such as glycerol, propylene glycol and polyethylene glycol, phthalate derivatives, citric esters, surfactants such as polyoxyethylene (20) sorbitan esters, and acetylated monoglycerides.

Sensory attributes may also be incorporated to the insoluble non-woven substrates. Examples of such sensory attributes include, but are not limited to color, texture, pattern, and embossing of the substrate.

The water-insoluble substrate when laid flat, may cover an area that is from about 100 cm² to about 1000 cm², such as from about 200 cm² to about 500 cm², such as between about 200 cm² to about 360 cm².

The water-insoluble substrate may have various shapes, such as an angular shape (e.g., rectangular) or an arcuate shape such as circular or oval.

The extracts and substrates/compositions containing such extracts of the present invention may be prepared using methodology that is well known by an artisan of ordinary skill.

### EXAMPLE 1

### Extract Preparations

The following is a description of the preparation of various extracts of the present invention. As used in the subsequent Examples, the weight percentage of extract refers to the weight of the liquid extract.

A: Malva Sylvestris Extract Preparation.

Malva sylvestris (whole dried flowers) was purchased from Botanic Choice (Hobart, Ind.) or Bilek (Troyan, Bulgaria). Ten grams of whole flowers were placed in 200 ml cold water, and brought to boiling in a sealed container. After the appearance of the boiling bubbles, the container was immediately withdrawn from the heating source, covered, and stored at room temperature for from about 1 hour to about 12 hours, with occasional agitation. The extract was then filtered through gauze, and excess liquid was squeezed manually from herbs to maximize the extract yield. The extract was either used as is or was further filtered through 22-micrometer 250 ml filtering unit from Nalgene (Rochester, N.Y.), under vacuum.

Alternatively, Malva sylvestris extract can be prepared by adding ten grams of whole flowers to 200 ml cold water, and agitating the mixture at room temperature for from about 1 hour to about 12 hours. The extract is then filtered as described above.

Alternatively, Malva sylvestris extract can be prepared by adding ten grams of whole flowers to 200 ml cold water, and then boiling the mixture in a sealed container. After the appearance of boiling, the container is withdrawn from the heating source, covered, and stored at room temperature for from about 1 hour to about 12 hours. After such time, ethanol is added to the extract to a final concentration of about 45%, volume of the total mixture. The extraction is continued at room temperature for additional 1 to 12 hours, with agitation. The extract was either used as is or was filtered as described above.

Alternatively, Malva sylvestris extract can be prepared as described above and dried. The evaporation is completed under helium lamp with a drying temperature of 100° C. The resulting dried powder is then resuspended to the original concentration with water (“Malva sylvestris reconstituted”). Filtration can be performed by passing the liquid through a 0.22 micrometer filter (Nalgene, Rochester, N.Y.). Using a HR73 Moisture Analyzer (Mettler-Toledo, Columbus, Ohio) to quantify the evaporating moisture at 30-second intervals, the dry content in the extracts prepared as described above was determined to be approximately 1.40% solids.

B: Cotinus Coggygria Extract Preparation.

Cotinus coggygria herb (whole dried leaf) was purchased from Bilkokoop (Sofia, Bulgaria). Ten grams of whole leaves were placed in 100 ml cold water, and brought to boiling in a sealed container, and boiled for 5 minutes. The container was then immediately withdrawn from the heating source, covered, and stored at room temperature for from about 1 hour to about 12 hours, with occasional agitation. After this, the extract was filtered through gauze, and excess liquid was squeezed manually from herbs to maximize the extract yield. The extract was either used as is or was further filtered through 22-micrometer 250 ml filtering unit from Nalgene (Rochester, N.Y.), under vacuum.

Alternatively, Cotinus coggygria extract is prepared by drying the extracts prepared as described above. The evaporation is completed under helium lamp with a drying temperature of 100° C. The resulting dried powder is then resuspended to the original concentration with water (“Cotinus coggygria reconstituted”). Filtration can be performed by passing the liquid through a 0.22 micrometer filter (Nalgene, Rochester, N.Y.). Using a HR73 Moisture Analyzer (Mettler-Toledo, Columbus, Ohio) to quantify the evaporating moisture at 30-second intervals, the dry content in the extracts prepared as described above was determined to be approximately 2.39% solids.

C: Matricaria Chamomilla Extract Preparation

Matricaria chamomilla herb (whole dried flowers) was purchased from Bilek (Troyan, Bulgaria). Matricaria recutita herb (whole dried flowers) was purchased from Botanic Choice (Hobart, Ind.). Ten grams of whole flowers were placed in 200 ml cold water, and brought to boiling in a sealed container. After the appearance of the boiling bubbles, the container was immediately withdrawn from the heating source, covered, and stored at room temperature for from about 1 hour to about 12 hours, with occasional agitation. After this, the extract was filtered through gauze, and excess liquid was squeezed manually from herbs to maximize the extract yield. The extract was either used as is or was further filtered through 22-micrometer 250 ml filtering unit from Nalgene (Rochester, N.Y.), under vacuum.

Alternatively, Matricaria chamomilla extract is prepared by drying the extracts prepared as described above. The evaporation is completed under helium lamp with a drying temperature of 100° C. The resulting dried powder is then resuspended to the original concentration with water (“Matricaria chamomilla reconstituted”). Filtration can be performed by passing the liquid through a 0.22 micrometer filter (Nalgene, Rochester, N.Y.). Using a HR73 Moisture Analyzer (Mettler-Toledo, Columbus, Ohio) to quantify the evaporating moisture at 30-second intervals, the dry content in the extracts prepared as described above was determined to be approximately 2.07% solids.

D: Arctostaphylos uva-ursi Extract Preparation.

Arctostaphylos uva-ursi herb (whole dried leaf) was purchased from Bilkokoop (Sofia, Bulgaria). Ten grams of whole leaves were placed in 100 ml cold water, and brought to boiling in a sealed container, and boiled for 5 minutes. The container was then immediately withdrawn from the heating source, covered, and stored at room temperature for from about 1 hour to about 12 hours, with occasional agitation. After this, the extract was filtered through gauze, and excess liquid was squeezed is manually from herbs to maximize the extract yield. The extract was either used as is or was further filtered through 22-micrometer 250 ml filtering unit from Nalgene (Rochester, N.Y.), under vacuum.

Alternatively, Arctostaphylos uva-ursi extract is prepared by drying the extracts prepared as described above. The evaporation is completed under helium lamp with a drying temperature of 100° C. The resulting dried powder is then resuspended to the original concentration with water (“Arctostaphylos uva-ursi reconstituted”). Filtration can be performed by passing the liquid through a 0.22 micrometer filter(Nalgene, Rochester, N.Y.). Using a HR73 Moisture Analyzer (Mettler-Toledo, Columbus, Ohio) to quantify the evaporating moisture at 30-second intervals, the dry content in the extracts prepared as described above was determined to be approximately 3.08% solids.

E: Thymus serpyllum Extract Preparation.

Thymus serpyllum herb (dried stem) was purchased from Bilek (Troyan, Bulgaria). Ten grams of whole herb were placed in 200 ml cold water, and brought to boiling in a sealed container. After the appearance of the boiling bubbles, the container was immediately withdrawn from the heating source, covered, and stored at room temperature for from about 1 hour to about 12 hours, with occasional agitation. The extract was then filtered through gauze, and excess liquid was squeezed manually from herbs to maximize the extract yield. The extract was either used as is or was further filtered through 22-micrometer 250 ml filtering unit from Nalgene (Rochester, N.Y.), under vacuum.

Alternatively, Thymus serpyllum extract is prepared by drying the extracts prepared as described above. The evaporation is completed under helium lamp with a drying temperature of 100° C. The resulting dried powder is then resuspended to the original concentration with water (“Thymus serpyllum reconstituted”). Filtration can be performed by passing the liquid through a 0.22 micrometer filter (Nalgene, Rochester, N.Y.). Using a HR73 Moisture Analyzer (Mettler-Toledo, Columbus, Ohio) to quantify the evaporating moisture at 30-second intervals, the dry content in the extracts prepared as described above was determined to be approximately 2.38% solids.

F: Herbal Combination Extract Preparation

Malva sylvestris herb (whole dried flowers) was purchased from both Bilek (Troyan, Bulgaria) or Botanic Choice (Hobart, Ind.). Matricaria chamomilla herb (whole dried flowers) was purchased from Bilek (Troyan, Bulgaria). Matricaria recutita was purchased from Botanic Choice (Hobart, Ind.). Thymus serpyllum herb (dried stem) was purchased from Bilek (Troyan, Bulgaria). Cotinus coggygria herb (whole dried leaf) was purchased from Bilkokoop (Sofia, Bulgaria). Thymus vulgaris herb (dried stem) was purchased from Starwest Botanicals (Rancho Cordova, Calif.). Amounts of herbs, as described in Tables 1, 2, and 3 below, were placed together in 250 ml cold water and brought to boiling in a sealed container. After the appearance of the boiling bubbles, the container was immediately withdrawn from the heating source, covered, and stored at room temperature for from about 1 hour to about 12 hours with occasional agitation. The extract was then filtered through gauze, and excess liquid was squeezed manually from herbs to maximize the extract yield. The extract was either used as is or was further filtered through 22-micrometer 250 ml filtering unit from Nalgene (Rochester, N.Y.), under vacuum.

Using a HR73 Moisture Analyzer (Mettler-Toledo, Columbus, Ohio) to quantify the evaporating moisture at 30 second intervals, the dry content in the extracts prepared as described above, was determined to be approximately 2% solids.

TABLE 1

| Name | Amount | |
|---|---|---|
| Malva sylvestris L. | 4 | g |
| Thymus serpyllum | 7 | g |
| Matricaria chamomilla L. | 7 | g |
| Water | 250 | ml |

TABLE 2

| Name | Amount | |
|---|---|---|
| Malva sylvestris L. | 4 | g |
| Thymus vulgaris | 7 | g |
| Matricaria recutita L. | 7 | g |
| Water | 250 | ml |

TABLE 3

| Name | Amount | |
|---|---|---|
| Malva sylvestris L. | 4 | g |
| Cotinus coggygria | 2.2 | g |
| Matricaria chamomilla L. | 7 | g |
| Water | 250 | ml |

Alternatively, the individual extracts of the herbs are separately made (e.g., as described in Examples 1A-1E) and subsequently combined together at a desired proportion (“Extract combined”). Added a newer description of the extracts
G. Soybean Extract Preparation

160 g of soybean powder (Sunlight Foods, Taipei, Taiwan) was added to about 1440 g of deionized water. The mixture was stirred at room temperature for about 1 hour. The mixture was then filtered through a sieve having holes of 75 μm diameter. The filtrate resulted in about 1.1 kg of soymilk.

### EXAMPLE 2

### Enhancement of Elastin Promoter Activity

Rat cardiac myoblasts H9C2 were purchased from ATCC (Manassas, Va.). Cultures were maintained in Dulbecco's modified Eagle's medium (DMEM, Invitrogen Life Technologies, Carlsbad, Calif.) supplemented with 10% fetal bovine serum, 2 mM glutamine, 100 units/ml penicillin, and 50 μg/ml streptomycin (Invitrogen life technologies, Carlsbad, Calif.).

Cell cultures were transiently transfected with the elastin promoter-luciferase reporter construct (Elp2.2, a 2.2 kb elastin promoter fragment from nt −2267 to nt +2, driving the firefly luciferase gene, which was obtained from Promega, Madison Wisc.). DNA was prepared by Qiagen Maxi columns (Qiagen Valencia, Calif.). In all transfections, a construct with the thymidine kinase promoter and the Renilla luciferase reporter gene (pRL-TK, Promega, Madison Wisc.) was included as an internal control. Cells were plated at 4×10⁴ in each well of a 24-well plate (Corning Incorporated, Corning, N.Y.) in growth media without antibiotics for 24 hours, reaching 80-90% confluency at the time of transfection. Typically, cells were transfected with 0.8 μg DNA per well using Lipofectamine 2000 (Invitrogen life technologies, Carlsbad, Calif.). One day after transfection, cells were treated with agents at indicated concentrations for approximately 48 hours before they were lysed for luciferase assays, using Dual-Luciferase Reporter System from Promega (Madison, Wisc.) following manufacture's protocol. Briefly, the firefly luciferase activity was measured first (representing elastin promoter activity), followed by the renilla luciferase (internal control), using luminometer LMAX, from Molecular Devices (Sunnyvale, Calif.). The ratio of these two luciferase activities (RLU) was used to evaluate the activity of each promoter.

Cells were treated with various doses of one or more of the following extracts: Malva Sylvestris extract (Example 1A), Coggygria extract (Example 1B), Matricaria chomomilla extract (Example 1C), Arctostaphylos uva-ursi extract (Example 1D), M. sylvestris/M. chamomilla/Thymus serpyllum extract (Example 1F), M. sylvestris/M. chamomilla/cotinus coggygria (Example 1F) or M. sylvestris/M. recutita/Thymus vulgaris extract (Example 1F), and Soybean Extract (Example 1G), and the effect of the extract on the induction of expression from the elastin promoter was evaluated. The extracts were added to the transfected H9C2 cells and were incubated for 48 hours. An increase in elastin promoter activity was observed in the presence of increasing doses of the extracts, as compared to untreated cells, as shown in Table 4. This example demonstrates that each of the extracts could enhance elastin production.

TABLE 4

| Agent (% W/W) | Induction |
|---|---|
| Control - no extract added | 1 +/− 0 |
| Malva sylvestris (2.5%) | 1.93 +/− 0.33 |
| Malva sylvestris (5%) | 2.27 +/− 0.03 |
| Cotinus coggygria (0.05%) | 1.75 +/− 0.52 |
| Cotinus coggygria (0.1%) | 1.62 +/− 0.3 |
| Cotinus coggygria (0.15%) | 1.5 +/− 0 |
| Matricaria chamomilla (5%) | 1.65 +/− 0.25 |
| Arctostaphylos uva-ursi (2.5%) | 1.56 +/− 0.34 |
| Malva sylvestris (5%) and Cotinus | 2.7 +/− 0 |
| coggygria (0.1%) | |
| Malva sylvestris (2.5%) and | 2.9 +/− 0.56 |
| Arctostaphylos uva-ursi (2.5%) | |
| Cotinus coggygria (0.05%) and | 2.27 +/− 0 |
| Arctostaphylos uva-ursi (2.5%) | |
| Malva sylvestris/Matricaria | 1.66 +/− 0 |
| recutita/Thymus vulgaris (2%) | |
| Malva sylvestris/Matricaria | 2.2 +/− 0 |
| chamomilla/Thymus serpyllum (2%) | |
| Malva sylvestris/Matricaria | 3.3 +/− 0 |
| chamomilla/Th. Vulgaris (2%) and Cotinus | |
| coggygria (0.15%) | |
| Malva sylvestris/ Matricaria chamomilla/ | 1.4 +/− 0.1 |
| Cotinus coggygria (2.5%) | |
| Malva sylvestris (0.77%) and Matricaria | 2.38 +/− 0.36 |
| chamomilla (1.35%) and Cotinus coggygria | |
| (0.38%) | |
| Malva sylvestris (1.54%) and Matricaria | 3.39 +/− 0.14 |
| chamomilla (2.7%) and Cotinus coggygria | |
| (0.76%) | |
| Soybean Extract (0.1%) | 1.36 +/− 0.2 |
| Soybean Extract (0.2%) | 1.65 +/− 0.15 |
| Soybean Extract (0.1%) and | 3.68 +/− 0.3 |
| Arctostaphylos uva-ursi (2.5%) | |

### EXAMPLE 3

### Protection from Elastase Degradation

Human leukocyte elastase (HLE) was purchased from Sigma (St. Louis, Mo.), and reconstituted at 1 unit/ml in phosphate buffered saline (PBS, Invitrogen life Technologies, Carlsbad, Calif.). Soluble bovine neck ligament elastin labeled with BODIPY FL dye was purchased from Molecular Probes, Inc. (Eugene, Oreg.), such that the fluorescence was quenched in the conjugate, and could be activated upon elastase digestion. Human leukocyte elastase (0.0625 U/ml), elastin substrate (25 μg/ml), and increasing concentrations of test material were incubated for one hour at room temperature. Fluorescence was measured at excitation at 490 nm and emission at 520 nm using a fluorescent plate reader Gemini from Molecular Devices (Sunnyvale, Calif.). Background fluorescence of substrate alone had been subtracted from each measurement.

Two batches of Cotinus coggygria extracts, prepared according to Example 1B, were averaged in the experiment, with data presented as compared to controls with no extract added. Cotinus coggygria extracts inhibited HLE activity in a dose dependent manner as shown in Table 5. As low as 0.01% of Cotinus coggygria extract resulted in approximately 60% reduction in HLE activity, while 0.1% of extract almost completely inhibited elastase activity. This example demonstrates that Cotinus extract can protect elastin fibers from damage and degradation.

TABLE 5

| Cotinus Extract (% W/W) | HLE Inhibition (%) |
|---|---|
| 0 | 0 +/− 1.6 |
| 0.0001 | 2.8 +/− 1.2 |
| 0.001 | 15.35 +/− 5.85 |
| 0.01 | 50 +/− 15 |
| 0.1 | 97.6 +/− 0 |

Soybean extracts, prepared according to Example 1F, were also used in the experiment, with data presented as compared to controls with no extract added in Table 6. Soybean extract inhibited HLE activity in a dose dependent manner (i.e., 0.0125% of Soybean extract resulted in approximately 45% reduction in HLE activity, while 0.1% of extract almost completely inhibited elastase activity). This example demonstrates that Soybean extract can protect elastin fibers from damage and degradation.

TABLE 6

| Soybean Extract (% W/W) | HLE Inhibition (%) |
|---|---|
| 0 | 0 +/− 6 |
| 0.0125 | 45 +/− 7 |
| 0.025 | 61 +/− 3 |
| 0.05 | 75 +/− 3 |
| 0.1 | 84 +/− 1 |

Human macrophage elastase (HME, also named Matrix Metalloproteinase-12, MMP-12) and fluorescently labeled substrate were purchased from R&D Systems (Minneapolis, Minn.). The fluorescence was quenched in the substrate, and could be activated upon elastase digestion. HME (100 ng/ml), substrate (10 μg/ml), and increasing concentrations of test material were incubated for one hour at room temperature. Fluorescence was measured at excitation at 320 nm and emission at 405 nm using a fluorescent plate reader Gemini from Molecular Devices (Sunnyvale, Calif.). Background fluorescence of substrate alone had been subtracted from each measurement.

Two batches of Cotinus coggygria extracts, prepared according to Example 1B, were averaged in the experiment, with data presented as compared to controls with no extract added. Cotinus coggygria extracts inhibited HME activity in a dose dependent manner as shown in Table 7. As low as 0.01% of Cotinus coggygria extract resulted in approximately 40% reduction in HME activity, while 0.5% of extract almost completely inhibited HME activity. This example demonstrates that Cotinus extract can protect elastin fibers from damage and degradation.

TABLE 7

| Cotinus Extract (% W/W) | HME Inhibition (%) |
|---|---|
| 0 | 0 |
| 0.01 | 37.6 +/− 2.4 |
| 0.05 | 69.6 +/− 1.0 |
| 0.1 | 79.5 +/− 1.5 |
| 0.5 | 96.3 +/− 0.4 |

Malva extracts, prepared according to Example 1A, were tested in the experiment, with data presented as compared to controls with no extract added. Malva extract inhibited HME activity in a dose dependent manner as shown in Table 8. As low as 0.6% of Malva extract resulted in approximately 23% reduction in HME activity, while 5% of extract inhibited HME activity 80%. This example demonstrates that Malva extract can protect elastin fibers from damage and degradation.

TABLE 8

| Malva Extract (% W/W) | HME Inhibition (%) |
|---|---|
| 0 | 0 |
| 0.6 | 22.0 +/− 0.9 |
| 1.25 | 40.1 +/− 0.0 |
| 2.5 | 62.0 +/− 0.6 |
| 5 | 79.3 +/− 1.2 |

Arctostaphylos uva-ursi extracts, prepared according to Example 1D, were tested in the same experiment, with data presented as compared to controls with no extract added. Arctostaphylos uva-ursi extract inhibited HME activity in a dose dependent manner as shown in Table 10. As low as 0.01% of Arctostaphylos uva-ursi extract resulted in approximately 10% reduction in HME activity, while 0.5% of extract inhibited HME activity 90%. This example demonstrates that Arctostaphylos uva-ursi extract can protect elastin fibers from damage and degradation.

TABLE 9

| Arctostaphylos uva-ursi | |
|---|---|
| Extract (% W/W) | HME Inhibition (%) |
| 0 | 0 |
| 0.01 | 10.8 +/− 2.0 |
| 0.05 | 44.9 +/− 0.4 |
| 0.1 | 62.4 +/− 1.8 |
| 0.5 | 89.5 +/− 0.5 |

Soybean extracts, prepared according to Example 1F, were used in the experiment, with data presented as compared to controls with no extract added. Soybean extract inhibited HME activity in a dose dependent manner as shown in Table 9. As low as 0.05% of Soybean extract resulted in approximately 22% reduction in HME activity, while 0.1% of extract showed 40% inhibition of HME activity. This example demonstrates that Soybean extract can protect elastin fibers from damage and degradation.

TABLE 10

| Soybean Extract (% W/W) | HME Inhibition (%) |
|---|---|
| 0 | 0 |
| 0.01 | 0 |
| 0.05 | 22.4 +/− 1.4 |
| 0.1 | 40.9 +/− 0.4 |

### EXAMPLE 4

### Enhancement of Elastic Fiber Network in Mouse Blood Vessel Walls

C57BL/6 female mice of age five (5) weeks were purchased from Taconic Farms (Germantown, N.Y.). Mice were housed in appropriately sized cages in an environmentally controlled room with a 12-hour light/12-hour dark photoperiod and supplied with food and water ad libitum. Animals were acclimated for three weeks before commencing the study. The animals were fed a special Casein Based Diet (5K96 with low isoflavone content, purchased from TestDiet (Richmond, Ind.) and housed together to achieve synchronized estrous cycling. After acclimation, 200 microliters of the unfiltered extract blend of Example 1E (Table 3) were given orally, once daily, five days a week (Monday through Friday). Skin and

Five months after the start of the ingestible treatments, bladder samples were obtained for histological analysis, and bladder blood vessels were analyzed histologically for elastin fibers. Elastin fiber density around blood vessels within the bladder sections was evaluated. As shown in Table 10, there was a considerable increase in the elastin fiber density in and around blood vessels in bladders of treated mice, as compared to controls.

Results of 22-week treatment are shown in Table 11 using the following grading: +=normal, ++=slightly increased, +++=moderately increased, and ++++=strongly increased.

TABLE 11

| | Elastin | |
|---|---|---|
| | in blood | |
| | vessels | N |
| Control | + | 2 out of 2 |
| Soybean Extract 5% | +++ | 3 out of 3 |
| Isoflavones 10% | ++++ | 3 out of 3 |
| Malva sylvestris/ | +++ | 3 out of 3 |
| Matricaria chamomilla/ | | |
| Cotinus coggygria | | |

### EXAMPLE 5

### Inhibition of Cytokine Secretion by Human Peripheral Blood Lymphocytes (PBL)

Agents were evaluated in vitro for their ability to inhibit the activation of PHA (Phytohemagglutinin) stimulated human peripheral blood lymphocytes (PBLs). Upon stimulation with PHA, mature T cells respond by clonal expansion and the secretion of cytokines. Helper T cells are subdivided into TH₁ and TH₂ subsets based on the spectrum of cytokines that they secrete. TH₁ cells produce IFNγ and IL-2, as well as GM-CSF, TNFα and TNFβ mediating macrophage activation as well as delayed hypersensitivity reactions. TH₂ cells secrete GM-CSF, IL-2, IL-4, IL-5, IL-6, IL-10 and IL-13 and are important in the regulation of humoral immunity, antibody responses and allergic reactions.

PBLs were collected from a healthy adult male and plated at 1×10⁶ cells/ml in Serum Free Lymphocyte Growth Media (ExVivo-15, BioWhittaker; Walkersville, Md.) and 100 μl were added to a flat bottomed 96 well plate. Human PBLs were stimulated with 10 μg/ml PHA (Remel, Lenexa, Kans.) in the presence or absence of test sample. PBLs were incubated at 37° C. at 5% CO₂ for 48 hours at which time supernatants were collected and stored at −20° C. until assayed. Cytokines were analyzed using commercially available multiplex kits (Upstate, Charlottesville, Va.) on a Luminex L100 (Luminex Corporation, Austin, Tex.). LDH (Boehringer Mannheim, Indianapolis, Ind.) was also run on the supernatants in order to determine viability. After supernatant was removed from part of the 96 well plate, the remaining cells were returned to the incubator and proliferation was determined at 72 hours by using alamar Blue (Alamar Bioscience, Sacramento, Calif.).

The composition from Example 1F (Malva sylvestris/Matricaria chamomilla/Cotinus coggygria) was tested in the human PBL assay, and cytokine secretion in response to PHA activation was measured. Unexpectedly, a dose-dependent inhibition of the secretion of all four cytokines tested was observed in the presence of the extract (Table 11). This predicts anti-inflammatory potential of the extract.

TABLE 12

| | % Inhibition | | | |
|---|---|---|---|---|
| Weight % | TNF-α | IL-2 | IFN-γ | IL-4 |
| 0.5% | 18% | 25.80% | 42.50% | 20% |
| 1% | 34.90% | 53% | 66.90% | 77% |
| 2.5% | 71.50% | 92.60% | 91.80% | 100% |

What is claimed is:

1. A method for treating hemorrhoids, said method comprising topically administering to tissue in need of such treatment a composition comprising cotinus coggygria extract.

2. A method of claim 1, wherein said composition further comprises malva sylvestris extract or soybean extract.

3. A method of claim 1, wherein said composition further comprises matricaria chamomilla extract.

4. A method of claim 2, wherein said composition further comprises matricaria chamomilla extract.

5. A method of claim 1, wherein said composition comprises from about 0.1%, by weight, to about 20%, by weight, of said cotinus coggygria extract.

6. A method of claim 5, wherein said composition further comprises from about 0.1%, by weight, to about 20%, by weight, of said Malva sylvestris extract or soybean extract.

7. A method of claim 5, wherein said composition further comprises from about 0.1%, by weight, to about 20%, by weight, of said matricaria chamomilla extract.

8. A method of claim 6, wherein said composition further comprises from about 0.1%, by weight, to about 20%, by weight, of said matricaria chamomilla extract.

9. A method of claim 1, wherein said composition further comprises one or more extracts selected from the group consisting of arctostaphylos uva-ursi extract, thymus vulgaris extract, thymus serpyllum extract, and matricaria recutita extract.

10. A method of claim 1, wherein said composition further comprises one or more of the following compounds selected from the group consisting of tocopherol or an ester thereof, live yeast cell extract, ephedrine, epinephrine, phenylephrin, witch hazel, mineral oil, petrolatum, shark liver oil, glycerin, kaolin, lanolin, wood alcohol, zinc oxide, shark liver oil, pramoxine, and hydrocortisone.

11. A method of claim 1, wherein said composition is in the form of a suppository.

12. A method of claim 1, wherein said composition is in the form of a lotion, cream, gel, or ointment.

13. A method of claim 1, wherein the composition is administered with a water-insoluble substrate comprising a non-woven, fibrous material.

14. A method of claim 2, wherein said composition is in the form of a suppository.

15. A method of claim 2, wherein said composition is in the form of a lotion, cream, gel, or ointment.

16. A method of claim 2, wherein the composition is administered with a water-insoluble substrate comprising a non-woven, fibrous material.

17. A method of claim 5, wherein said composition is in the form of a suppository.

18. A method of claim 5, wherein said composition is in the form of a lotion, cream, gel, or ointment.

19. A method of claim 5, wherein the composition is administered with a water-insoluble substrate comprising a non-woven, fibrous material.

20. A method of claim 6, wherein said composition is in the form of a suppository.

21. A method of claim 6, wherein said composition is in the form of a lotion, cream, gel, or ointment.

22. A method of claim 6, wherein the composition is administered with a water-insoluble substrate comprising a non-woven, fibrous material.
